(19) United States
(12) Patent Application Publication
(10) Pub. No.: US 2008/0317717 A1
(43) Pub. Date: Dec. 25, 2008

(54) ANTI-HEPATITIS B VIRUS RIBOZYMAL NUCLEIC ACID

(75) Inventors: Peter Anthony Minter EAGLES, London (GB); Amer Qureshi, London (GB)

Correspondence Address: BANNER & WITCOFF, LTD., 28 STATE STREET, 28th FLOOR, BOSTON, MA, 02109-9601, US

(73) Assignee: King's College London, London (GB)

(21) Appl. No.: 12/112,590

(22) Filed: Apr. 30, 2008

Related U.S. Application Data
(63) Continuation of application No. PCT/GB2006/004114, filed on Nov. 3, 2006.

(30) Foreign Application Priority Data
Nov. 4, 2005 (GB) 0522578.4

(51) Int. Cl. A61K 35/76; C07H 21/04; C12N 15/63; A61P 31/12; A61K 31/7052; C12N 7/01

(52) U.S. Cl. 424/93.2; 536/23.1; 435/320.1; 435/235.1; 514/44

(57) ABSTRACT

This invention relates to ribozymes which cleave Hepatitis B Virus (HBV) at CUC sites. Suitable ribozymes may, for example, cleave at GGCUCUCUCGUCCC, CCUCAGCUCUGUAUCG or GAGGACUCUUGGA recognition sequences in HBV RNA. Ribozymal DNA, vector systems and pharmaceutical compositions are provided which may be useful, for example, in the treatment of HBV infection.

### RELATED APPLICATIONS

This application is a continuation of PCT application no. PCT/GB2006/004114, designating the United States and filed Nov. 3, 2006; which claims the benefit of the filing date of United Kingdom application no. GB 0522578.4, filed Nov. 4, 2005; each of which is hereby incorporated herein by reference in its entirety for all purposes.

### FIELD OF THE INVENTION

This invention is in the field of recombinant DNA technology and is more particularly concerned with Ribozyme technology, especially with the design of ribozymes against Hepatitis B Virus (HBV).

### BACKGROUND OF THE INVENTION

Hepatitis B Virus (HBV) infects hundreds of millions people across the world. Effective preventive measures are available in the form of vaccination, but a highly potent therapy for HBV-infected people has long been awaited. The only hope a therapeutic intervention offers to a HBV patient is to reduce the progression of disease by interrupting the replication of HBV. There are many anti-HBV therapies which have been proposed, and which give some relief from the disease, but none provides efficient help in the battle against the virus.

HBV genome consists of a circular, and partially double stranded DNA of 3.2 kb. HBV uses pregenomic RNA for its replication. The pre-genomic RNA is characterized as a 3.5 kb sequence, longer than the genome (3.2 kb), which is reverse transcribed by the viral polymerase. Both ends of this RNA have a unique stem-loop structure composed of 60 bases, called the ε loop. The 5′ end ε-loop is known to play an essential role in encapsidation of the pregenomic RNA and it also provides a site for the polymerase to bind prior to reverse transcription.

The use of recombinant DNA/RNA to modulate expression of genes and targeting specifically viral genomes to stop their replication has provided very efficient therapeutic tools. Ribozymes, which are catalytic RNA molecules to target specific sequences, have been used in the past for the depletion of HBV. Hammerhead ribozymes have been designed against HBV, depleting the pregenomic RNA and mRNA for its proteins. These ribozymes were designed to target three different sites on the HBV pregenomic RNA (von Weizsacker, et. al., 1992). Other workers designed hammerhead ribozymes to bind and cleave the e-loop region with modest results (Beck and Nassal, 1995). The RNA sequence coding for gene X along the HBV sequence has also been targeted using hammerhead ribozymes (Kim et. al., 1999, and Weinberg et. al., 2000).

The hammerhead ribozymes previously designed against HBV are not efficient in cleaving their target, due to their design and synthesis procedures. HBV ribozymes in accordance with the present invention have been shown to fully cleave the target HBV sequence in-vitro. The use of two or more ribozymes in accordance with the present invention results in cutting the target HBV messenger or pregenomic RNA at two or more different sites. Strictly, a ribozyme is an RNA molecule which cleaves an RNA target. Some of the literature is using the term to describe DNA molecules which are transcribed to RNA, thus generating the ribozyme proper. In this specification, the term “ribozymal DNA” means DNA transcribable to the ribozyme proper.

### SUMMARY OF THE INVENTION

Ribozymes of the present invention, and their corresponding ribozymal DNA molecules which are transcribed in vivo, are designed to cleave CUC sites in the HBV messenger RNA or pregenomic RNA. As is well known, the therapeutic use of ribozymes is achieved via administration of their counterpart DNA usually incorporated in vectors such as plasmid vectors.

The design and construction of ribozymes in accordance with the present invention, which will be described in detail hereinafter, is exemplified by the preparation of three hammerhead ribozymes herein named as MAZI, SGIII and DGII. The cleavage sites in the HBV mRNA at which these exemplary ribozymes act is shown in relation to the HBV sequence (expressed as cDNA) in Table 1.

The HBV sequence (AYW Strain) was obtained from GCG (Acc. No. Y07587), the sequence 500 bases upstream and 500 bases downstream of the ε-loop region (1848-1910). Computer program “mfold” (http://bioweb.pasteur.fr/seqanal/interfaces/mfold-simple.html) was used to predict secondary structure of HBV RNA to determine the free NUX sites, i.e. not involved in strong binding to form a strand, and to design the secondary structure of designed hammerhead ribozymes against the free NUX sites. The NUX sites selected were all CUC sites.

By way of example three trans-acting hammerhead ribozymes have been designed, all three of which comprise a trans-acting ribozyme as the effective cleaving agent for HBV RNA preceded by a stability loop at the 5′ end and followed by one or more additional stem loop structures. The trans-acting component will usually be followed by a cis-acting ribozyme at the 3′ end. Throughout this specification, reference will be frequently made to ribozymal design in terms of RNA nucleotides, these being the molecular species which are operative at the HBV RNA target sites. It will be appreciated however that the therapeutic agent administered in vivo to achieve cleavage of HBV RNA will require the formulation of the corresponding DNA polynucleotide(s) in an appropriate vector system.

In one aspect, the invention provides a vector system comprising at least one DNA vector, the vector or vectors containing a target-cleaving hammerhead ribozymal DNA sequence under control of a promoter effective in human cells and which, upon transcription to RNA will cleave the mRNA transcribed from a target gene encoding the HBV RNA at CUC cleavage sites.

The linkage of the ribozymal DNA sequence to the promoter can be direct, and need employ only a single vector. However, there are advantages in an indirect linkage which amplifies the effect of the promoter. Such an indirect linkage will normally require two or more vectors. Thus, the invention includes a vector system comprising at least two DNA vectors, wherein a first vector contains a first promoter effective in human cells, operably linked to a gene which is expressible to produce an activator protein capable of acting on a second promoter, and a second vector contains the second promoter operably linked to the target-cleaving hammerhead ribozymal DNA sequence referred to above. The ribozymal DNA sequence can comprise a composite sequence for cleaving the HBV RNA at two or more sites. The term “vector system” as used herein is generic terminology encompassing a single vector or a kit or composition or two or more vectors.

Further, the invention includes ribozymal DNA, both per se and as a ribozymal DNA sequence contained within a vector, the ribozymal DNA further comprising, downstream of the target-cleaving ribozymal sequence, a 3′-autocatalytic hammerhead ribozymal DNA sequence, so that when the ribozymal DNA is transcribed to RNA it has a form representable as a double hammerhead, having first and second stems of a target-cleaving ribozyme which targets HBV RNA and first and second stems of 3′-autocatalytic ribozyme, together with a common, third stem joining the two hammerheads. This third stem is preferably of at least 4 bases near the 3′ end of the HBV ribozyme sequences, capable of base-pairing with a complementary sequence of at least four bases near the 3′ end of the autocatalytic ribozyme sequence, so as to form, when base-paired, the said common stem joining the hammerheads of the target-cleaving and 3′-autocatalytic ribozymes.

The verb “to comprise,” whenever used herein in any grammatical form, means to consist of or include.

### BRIEF DESCRIPTION OF THE DRAWINGS

FIG. 1 depicts a target-cleaving ribozyme sequence of the invention for MAZI ribozyme.

FIG. 2 depicts a target-cleaving ribozyme sequence of the invention for SGIII ribozyme.

FIG. 3 depicts a target-cleaving ribozyme sequence of the invention for DGII ribozyme.

FIG. 4 shows the MAZI ribozyme aligned with HBV RNA in the vicinity of the cleavage site.

FIG. 5 shows the SGIII ribozyme aligned with HBV RNA in the vicinity of the cleavage site.

FIG. 6 shows the DGII ribozyme aligned with HBV RNA in the vicinity of the cleavage site.

FIG. 7 depicts the MAZI ribozyme combined with an autocatyltic ribozyme in a trans/cis double ribozyme.

FIG. 8 depicts the SGIII ribozyme combined with an autocatyltic ribozyme in a trans/cis double ribozyme.

FIG. 9 depicts the DGII ribozyme combined with an autocatyltic ribozyme in a trans/cis double ribozyme.

FIGS. 10, 11 and 12 are photographs of agarose gels with ethidium bromide, containing human HBV RNA and incubated with MAZI, SGIII and DGII ribozymes respectively corresponding to the ribozymal DNA sequence provided by a vector system of the invention; these photographs show how the HBV RNA has been cleaved after 4 hours of incubation.

FIG. 13 is a schematic diagram showing a 3-plasmid vector system of the invention, the first plasmid comprising a CMV promoter driving transcription of mRNA from a T7 polymerase gene, the second plasmid comprising a T7 promoter driving transcription of mRNA from a T7 polymerase gene and the third plasmid comprising a T7 promoter driving transcription of RNA from a ribozymal DNA cassettes which targets HBV RNA at three different sites.

FIG. 14 is a schematic drawing of the lentiviral vector packaging and envelope constructs. pCMVΔR8.91 (http://www.medecine.unige.ch/˜salmon/main/R891.gif) and pCMV-VSV-G (www.brc.riken.jp/lab/cfm/map/pCMV-VSV-G%20map.pdf)

FIG. 15 is a schematic drawing of the lentiviral vector gene transfer construct pHR cRT7HBVRz which contains an expression system for HBV ribozymes MAZI, DGII and SGIII. The T7 RNA Polymerase fused with a red fluorescent protein is expressed under a CMV promoter, and transcribes the MAZ, DG and SG ribozymes from their respective DNA cassettes cloned downstream. This vector is used in conjunction with packaging plasmids to make Lenti-HBVRz virus.

FIG. 16 is a schematic drawing of the HBV target sequence expression constructs. pHBV1 is a mammalian expression vector that contained the HBV genome fragment from 1-1579 (Accession no. Y07587). The cleavage site of MAZ I Rz is present in this vector. The pHBV2 also a mammalian expression vector contained the HBV fragment from 1500-3182 (Accession no. Y07587). The cleavage sites for ribozymes DG II and SG III are present in this vector.

FIG. 17 is a photograph of an agarose gel stained with ethidium bromide showing cleavage of HBV target sequences with lentivirally-delivered MAZI, SGIII and DGII.

### DESCRIPTION OF THE PREFERRED EMBODIMENTS

The ribozymes of this invention may be of the hammerhead type. Ribozymes have catalytic sequences which cleave the RNA at the desired target site. The catalytic sequences of hammerhead ribozymes are usually of the form (5′ to 3′) (1) cuganga . . . and (2) . . . gaa, where n is any nucleotide. In the present invention n is preferably u. They are separated by a stabilizing structure, which is preferably a stem loop. The ribozymal DNA in the invention can have this form (substituting thymine for uracil).

The most preferred target sequences in HBV RNA, for the purposes of the present invention, are

| | | | | |
|---|---|---|---|---|
| MAZI: | 5′ | ggcucucucguccc | 3′ | (SEQ ID NO: 1) |
| SGIII: | 5′ | ccucagcucuguaucg | 3′ | (SEQ. ID NO: 2) |
| DGII: | 5′ | gaggacucuugga | 3′ | (SEQ ID NO: 3) |

The underlined portion is the essential sequence of three bases required by the hammerhead ribozyme used in the present invention.

Immediately upstream and downstream of the catalytic sequences lie target-binding (i.e. target-recognition) sequences. The target is RNA, and the ribozyme which is RNA, is complementary to the target RNA, (disregarding the additional c nucleotide present in the target, as explained below).

The sequences involved in the preferred target and in the preferred ribozyme binding thereto may therefore be summarized as follows:

| | | |
|---|---|---|
| MAZI | | |
| (a) | 5′ ggcucu c* ucguccc 3′ | (target RNA) |
| (b) | 3′ ccgaga -cat.seq.-s.l.-cat seq.-agcaggg 5′ | (rz RNA) |
| (c) | 5′ ggctct -cat.seq.-s.l.-cat seq.-tcgtccc 3′ | (rz DNA; strand 1) |
| (d) | 3′ ccgaga -cat.seq.-s.l-cat seq. -agcaggg 5′ | (rz DNA; strand 2) |
| SGIII | | |
| (a) | 5′ ccucagcu c* uguaucg 3′ | (target RNA) |
| (b) | 3′ ggagucga-cat.seq.-s.l.-cat.seq -acauagc 5′ | (rz RNA) |
| (c) | 5′ cctcagct-cat.seq.-s.l.-cat.seq.-tgtatcg 3′ | (rz DNA; strand 1) |
| (d) | 3′ ggagtcga-cat.seq.-s.l.-cat.seq.-acatagc 5′ | (rz DNA; strand 2) |
| DGII | | |
| (a) | 5′ gaggacu c* uugga 3′ | (target RNA) |
| (b) | 3′ cuccuga-cat.seq.-s.l.-cat.seq.-aaccu 5′ | (rz RNA) |
| (c) | 5′ gaggct-cat.seq.-s.l.-cat.seq.-ttgga 3′ | (rz DNA; strand 1) |
| (d) | 3′ ctcctga-cat.seq.-s.l.-cat.seq.-aacct 5′ | (rz DNA; strand 2) |

(* = cleaved nucleotide, cat.seq. = catalytic site; s.l. = stem loop)

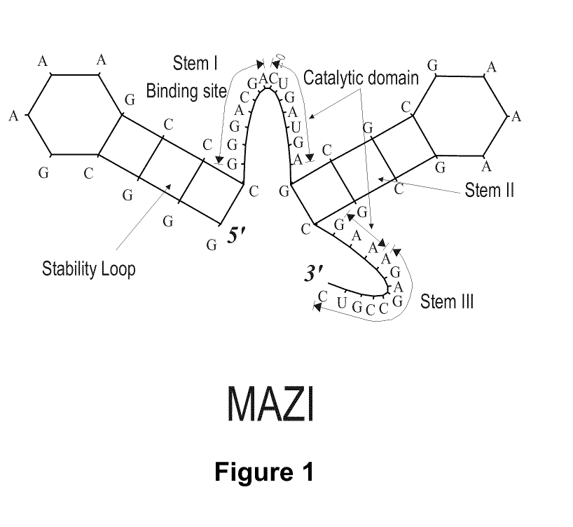

For the ribozyme MAZI targeting the HBV RNA, 5′ gaggacu 3′ is the first target-recognition sequence and 5′ ucguccc 3′ is the second target-recognition sequence. The cleavage site in the target is cuc*, the asterisked c nucleotide being the cleavage site and therefore having no counterpart in the ribozyme. Sequences (a) and (b) for MAZI ribozyme are shown in FIG. 1 of the drawings.

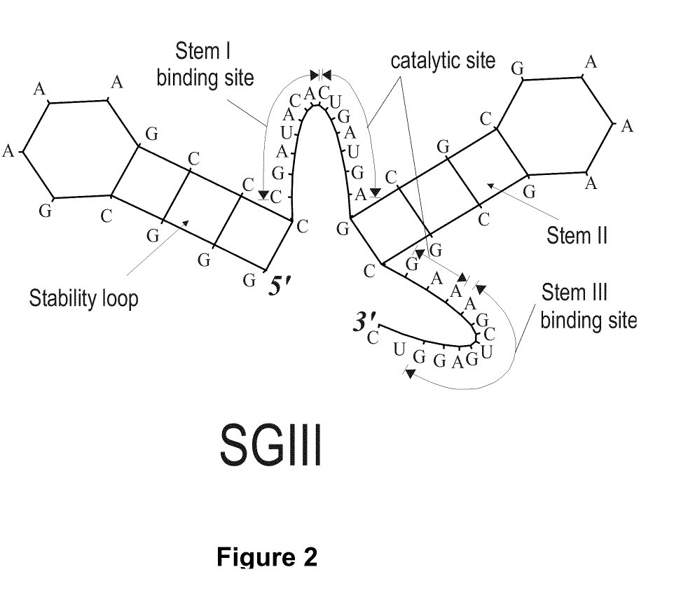

For the ribozyme SGIII targeting the HBV RNA, 5′ ccucagcu 3′ is the first target-recognition sequence and 5′ uguaucg 3′ is the second target-recognition sequence. The cleavage site in the target is cuc*, the asterisked c nucleotide being the cleavage site and therefore having no counterpart in the ribozyme. Sequences (a) and (b) for SGIII ribozyme are shown in FIG. 2 of the drawings.

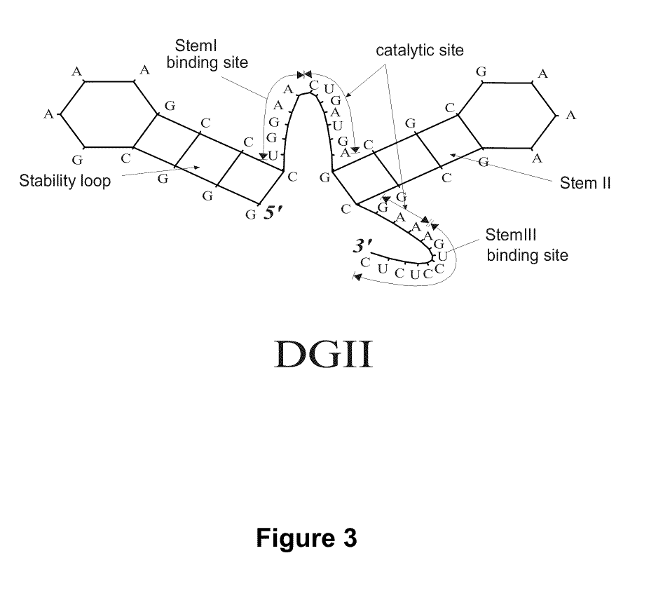

For the ribozyme DGII targeting the HBV RNA, 5′ gaggacu 3′ and 5′ uugga 3′ are the first and second target-recognition sequences. The cleavage site in the target is again cuc*. A preferred sub-genus of ribozyme for use in the invention is those which have these target recognition sequences. Sequences (a) and (b) for DGII ribozyme are shown in FIG. 3 of the drawings.

In the following description, the structure of hammerhead ribozymes is discussed in RNA terms, but it will be understood that the ribozymal DNA, from which they are transcribed, corresponds, substituting thymine for uracil. It will also be appreciated that the conformations of these ribozymes shown herein are those evident from base-pairing and other energetic considerations and that the invention is in no way limited by these drawings, i.e. that the invention includes other conformations of the same molecules.

Hammerhead ribozymes are maintained by two stem loops, a first stem loop (“stem I”) preceding the first target-recognition sequence and the second stem loop (“stem II”) lying between the first and second target-recognition sequences. These two stem loops may have any desired form, but typically comprise 3 to 5 complementary base pairs forming the stem and 4 or 5 bases in the loop.

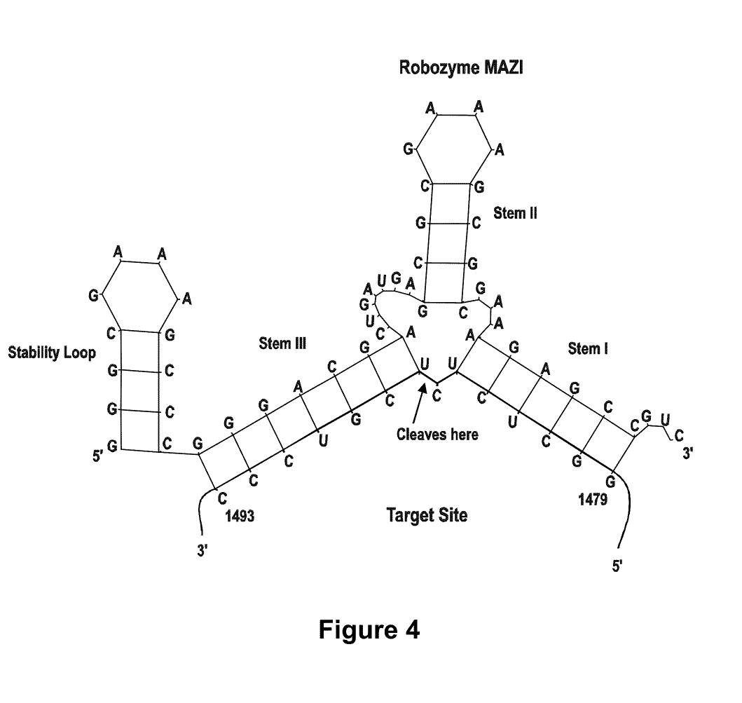

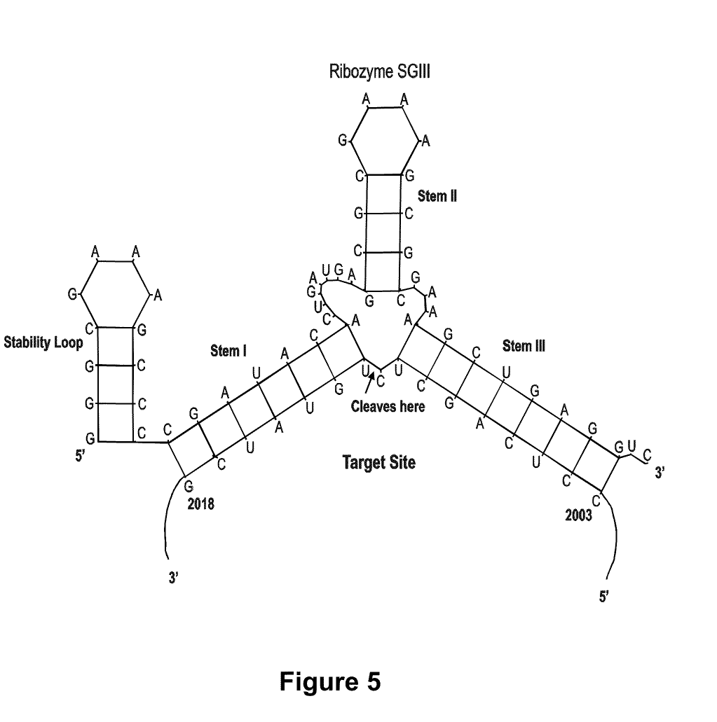

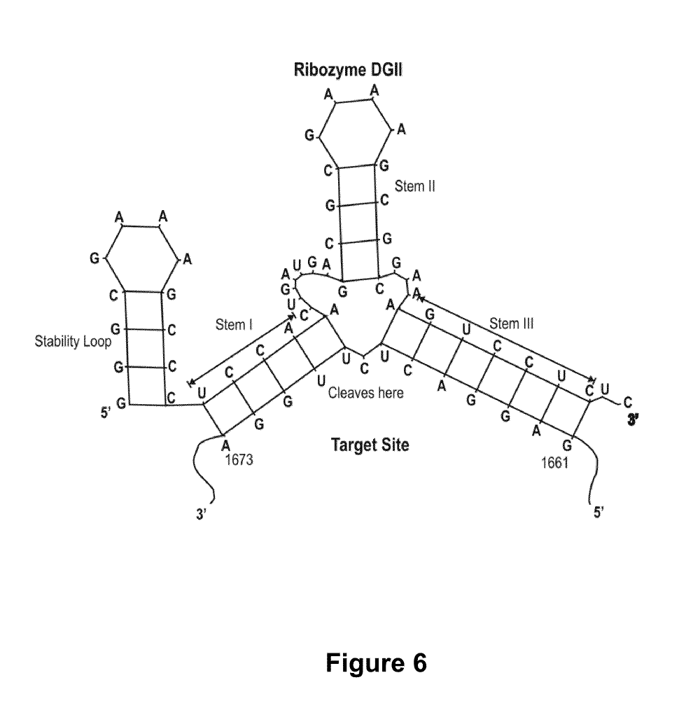

Following the second catalytic sequence is a third stem, which consists of or includes the second target-recognition sequence. In one embodiment of the invention, stem III has a special sequence of guc at the 3′ end of MAZI and SGIII which can be added in part or in full, if not naturally present. Where, as here, g is the natural ending in the case of SGIII uc is added as an over-hang where as in the case of MAZI guc is added as an overhang, so that the last few bases thereof pair with the bases of the second catalytic sequence. With DGII, c is added as an overhang. FIG. 4 shows MAZI binding to its target site, FIG. 5 shows SGIII attached to its target area and FIG. 6 shows DGII binding to its target site. These diagrams are achieved by a-u and g-c base pairs.

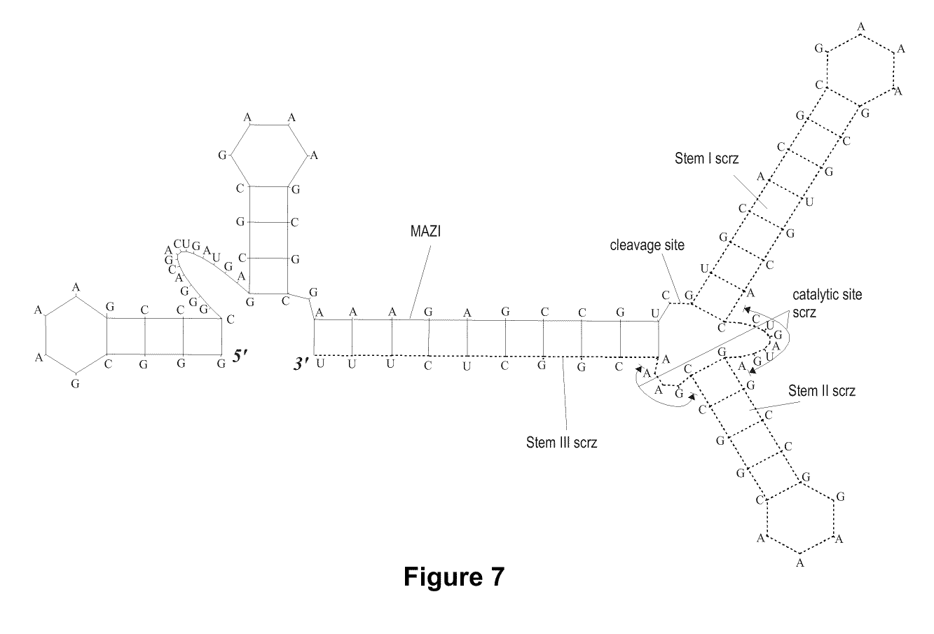

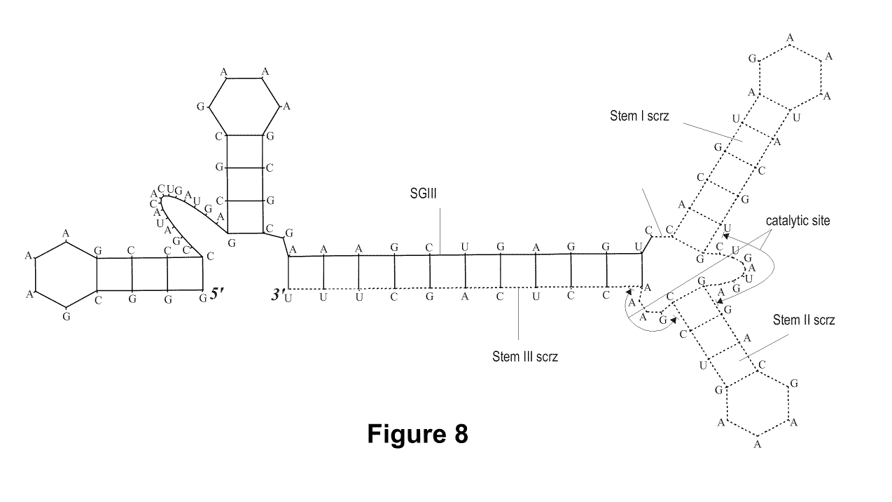

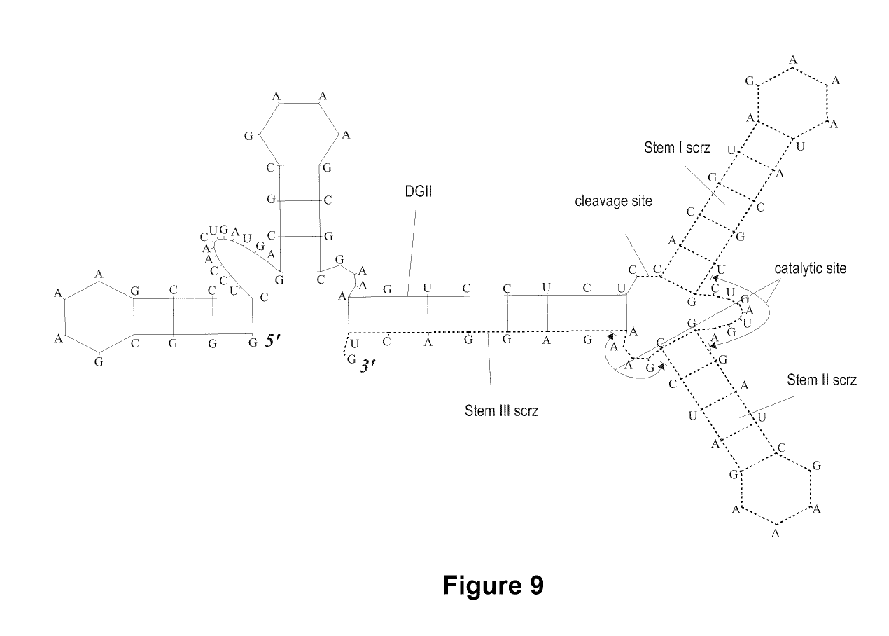

More preferably, the ribozyme contains a 3′-autocatalytic sequence. Such sequences are known per se, especially from PCT Patent Application Publication N^{o} WO 97/17433 (Medical University of South Carolina). The 3′-autocatalytic sequence is preferably designed so that a sequence near the 3′ end of the target-cleaving ribozyme is base-paired with a downstream part of the autocatalytic sequence at or close to the 3-end thereof. These preferred constructs have at least 4 base pairs in stem III. They may have as many as 10 of these base pairs. Typically the over-hang of non-base-paired nucleotides extending beyond stem III is only 1 or 2 at the 3′-end of the target cleaving ribozyme sequence and 0 to 5 at the 3′-end of the autocatalytic sequence. Such constructions are exemplified for MAZI in FIG. 7, SGIII in FIG. 8 and DGII in FIG. 9. Here the 3′-autocatylytic (=self-cleaving) sequence is in the form of a hammerhead ribozyme comprising a 3′-cleavage site (guc for MAZI and SGIII, cuc for DGII), a first stem loop, (“scrz Stem I”), a second stem loop (“scrz Stem II”) and a third stem (“scrz Stem III”), the third stem being base—paired with Stem III of the target-cleaving ribozyme. Cleavage occurs after the c of the guc 3′-cleavage site. The catalytic sequence (cugauga) between scrz stem I and scas stem II is one which assists in stabilizing the hammerhead structure and is also used in WO 97/17433. Between Stems II and III there is provided a gaa catalytic site.

The ribozymes used in the present invention may contain a 5′-autocatalytic sequence, for example as described in WO 97/17433. In WO 97/17433 a double ribozyme is provided containing a centrally located BglII cloning site agatct into which any desired target-recognition and catalytic sequences can be inserted. In WO 97/17433, the insert is of 42 bases. These consist of first target-recognition sequence (8 bases), catalytic and structure-stabilizing sequences (23 bases) and a second target-recognition sequence (11 bases, the last two of which are the ag of a BglII site). With minor modifications, the same construction could be adapted to the present invention, e.g. substituting the DNA equivalent of 36 bases, 13-48 of FIG. 1, for the 23 bases of WO 97/17433, adding nucleotides necessary for cloning into a BglII site at the 3′-end thereof. In this construction Stem I would be dispensed with and replaced by the 5′-sequence of WO 97/17433 including the 5′-autocatalytic site. However, in WO 97/17433 the portion of sequence between the catalytic sites is not in a tight stem loop form and so appears less structure-stabilizing.

### Engineering of the Polymerase Vector

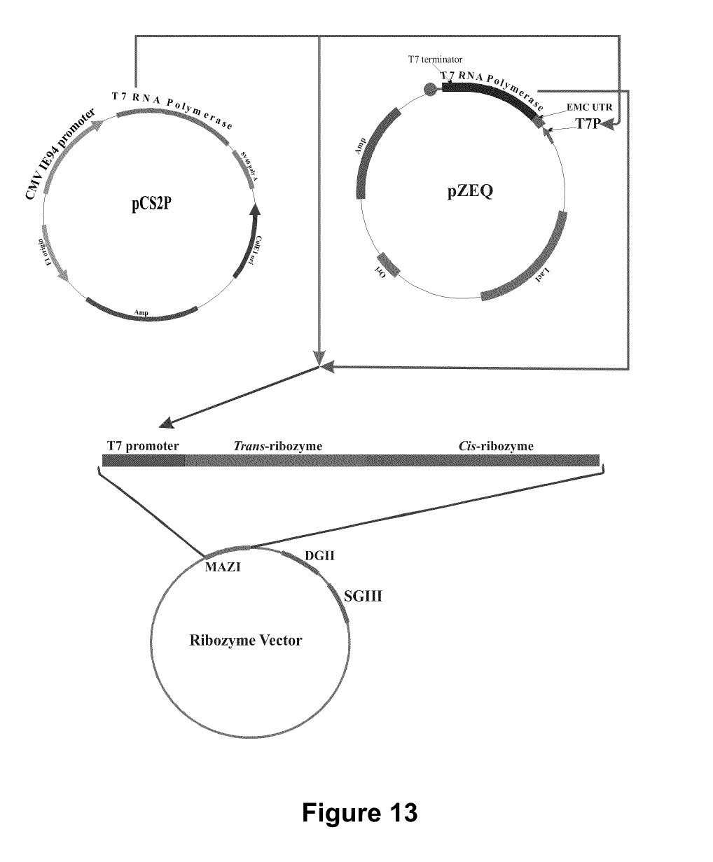

Referring to FIG. 13, the polymerase vector pCS2P comprises a promoter from cytomegalovirus (CMV) and the T7 polymerase gene. The complete T7 polymerase DNA sequence is available from Genbank/EMBL under Accession No. M. 38308. In this Example, a modified T7 polymerase DNA was obtained and amplified by PCR on plasmid pT7AutoI [J. Dubendorff and F. Studier, J. Mol. Biol. 219, 61-68 (1991)]. The primers used for the PCR incorporated the restriction sites EcoRI and NcoI at the 5′ end of the forward primer; and BamHI, at the 5′ end of the reverse primer. (BamHI was used later for the cloning of the autopolymerase vector.) The sequences of the primers were as follows with the overlapping T7 polymerase underlined.

Forward:
5′ acgaattccatggacacgattaacatcg 3′
EcoRI site = gaattc; NcoI site = ccatgg
Reverse:
5′ atataaggatccttacgcgaacgcgaac 3′
BamHI site = ggatcc

The PCR was carried out using Vent polymerase (which provided a ‘blunt end’ in the PCR product). The NcoI site introduced by the forward primer is an extra cloning site and was produced by changing the second codon of the T7 polymerase, DNA from an Asn (aac) to an Asp (gac). This does not change the activity of T7 polymerase.

The T7 polymerase PCR product was cloned into a pCS2-NLS plasmid (R. A. W. Rupp et al. Genes and Development 8, 1311-1323 (1994) and D. L. Turner & H. Weintraub ibid. 1434-1447]. The T7 polymerase DNA was introduced into an EcoRI site and a SnaB1 (blunt ended) site in the pCS2-NLS, located shortly after the NLS in the clockwise direction. The NLS sequence in the pCS2 was unnecessary for the present purpose at this stage and it was deleted by cutting with restriction enzyme NcoI as the NLS sequence was now in between the two NcoI sites. Then the plasmid was religated through the NcoI sites. Thus the polymerase plasmid vector was completed as shown in FIG. 13 (pCS2P). It contained a CMV promoter (already existing in the pCS2 vector), which switched on the production of T7 polymerase. T7 polymerase is required for the ribozymal DNA vector and the autopolymerase vector, detailed below.

### Engineering of the Autopolymerase Vector

The purpose of this vector was to provide a steady and adequate supply of T7 polymerase. The T7 promoter was used to switch on the production of T7 polymerase. This polymerase acted autocatalytically making more T7 promoter which made more T7 polymerase (FIG. 13).

mRNAs made by transfected vectors through non-mammalian promoters in mammalian cell cytoplasm are not usually recognised by the cells for translation into proteins. In order to trick the cell into translating the T7 polymerase mRNA transcribed by the promoter, an encephalomyocarditis (EMC) virus, UTR (untranslated region) sequence, (Moss et al., Nature 348, 91-92 (1990)) was added. This sequence serves as a translational enhancer, providing binding sites for ribosomes and was obtained by PCR-amplifying EMC UTR from the pTM1 vector; see B. Moss et al., Nature 348, 91-92 (1990). The primers used included an XbaI restriction site in the forward strand. No restriction site was introduced in the reverse primer, since the EMC sequence obtained from this vector contained several engineered restriction sites, such as BamHI and NcoI. The primers were as follows, with the overlapping EMC sequences underlined:

Forward:
5′ gctctagaccacaacggtttccctctag 3′
XbaI site = tctaga,
Reverse:
5′ cagcttcctttcgggctttgttagcagc 3′

The EMC sequence was then cloned into pETlla (Novagen Ltd.) using XbaI and BamHI sites. For a map, see e.g. the 1996/97 catalogue of R & D Systems Ltd., Abingdon, Oxfordshire, England. page 74. The EMC UTR sequence naturally contained a NcoI restriction site at its 3′ end, in front of a BamHI site. Thus, the T7 polymerase sequence, as described above in Section 6, which contained NcoI and BamHI sites, was readily cloned into the plasmid downstream of the EMC UTR sequence, as described by X. Chen et al., Nucleic Acids Research 22, 2114-2120 (1994). See e.g. FIG. 13, plasmid pZEQ.

Referring to FIG. 13, the ribozyme vector comprises ribozymal DNA encoding MAZI, DGII and SGIII. It has long been thought desirable to attack the HBV at more than one point in its cycle of infection, growth and replication. Thus the same vector could contain one or more other kinds of ribozymal DNA which will target other RNA produced by HBV or required to make a new HBV genome, which is vital for its growth or replication. Thus, FIG. 13 illustrates a vector containing three kinds of ribozymal DNA MAZI, DGII and SGIII.

In order to “kick start” the promoter it is desirable to provide a separate source of the polymerase, either as the enzyme itself or, more preferably in the form of another vector, which is also preferably a plasmid, dedicated for this purpose. See FIG. 13, plasmid pCS2P.

Referring to FIG. 13, pCS2P contains the CMV promoter driving transcription of the T7 polymerase gene, a second plasmid, pZEQ, contains the T7 autogene system for the production of T7 polymerase and a third plasmid in which a T7 promoter, activated by the polymerase produced by the first two plasmids, drives transcription of the HBV ribozymes incorporated in the ribozyme vector, shown in FIG. 13. Other translational enhancers could be used in place of that shown.

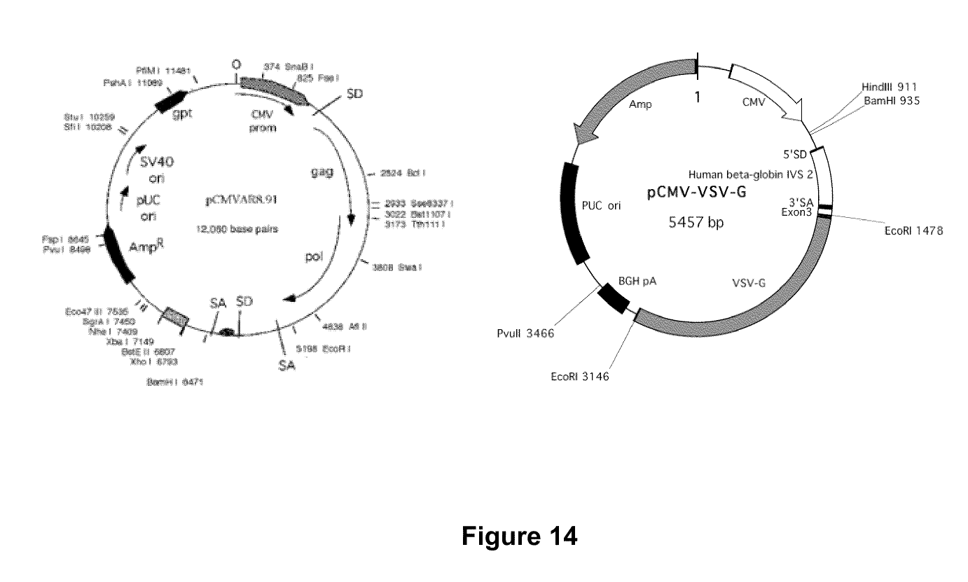

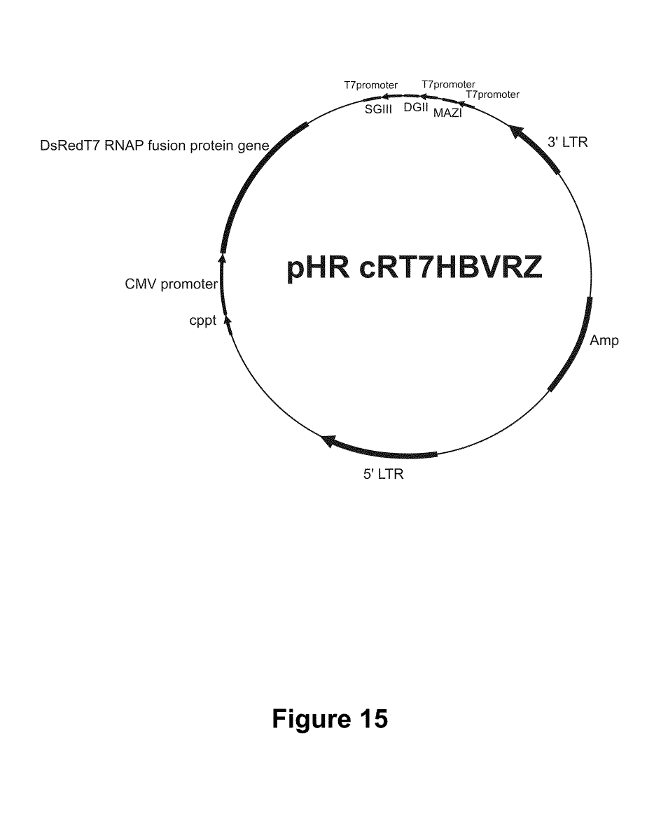

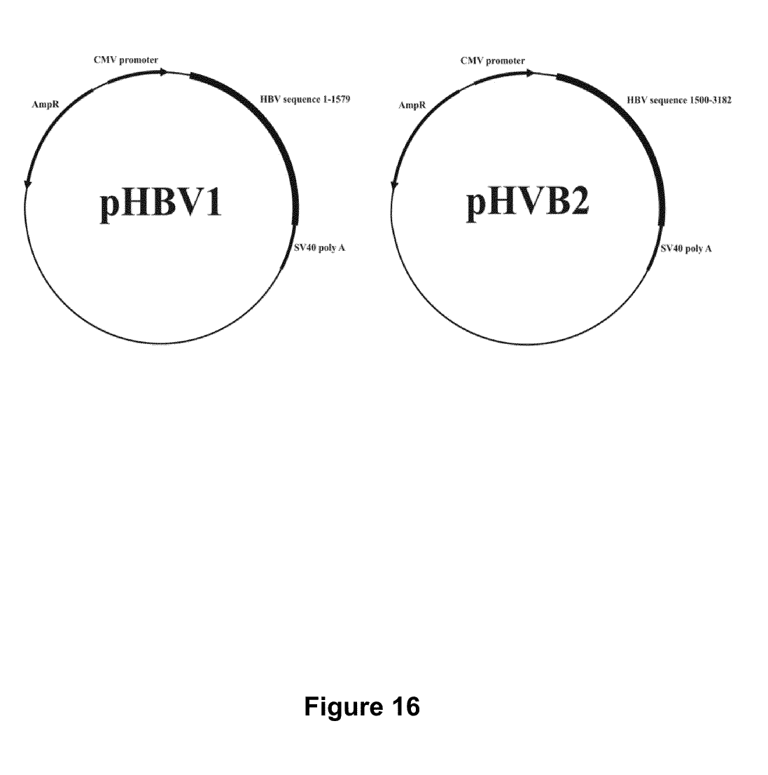

In some embodiments, lentiviral vectors are preferred. Lentiviral vectors derived from HIV offer long term stable in vitro and in vivo gene transfer. These vectors are attractive as they can transfer large transgenes (up to 18 kb) and are able to stably transduce both dividing and non-dividing cells. Such systems generally use a gutted HIV genome that prevents replication and in which viral genes responsible for infectivity and virulence are removed. In order to further increase the safety of the system and to reduce the possibility of the generation of replication competent retroviruses, lentiviral gene transfer vectors are generally assembled by the co-transfection of 3 plasmids into cell lines in vitro; the packaging construct, the envelope construct and the gene transfer construct. Such vectors may also be self-inactivating. See Zuffrey et al. J. Virology 72, 9873-9880 (1998). In order to widen the tropism of the vector and to improve its physiochemical properties, such vectors are often pseudo-typed with the envelope protein from the vesicular stomatitis virus (VSV-G). In this current embodiment, FIG. 14 shows the packaging construct, FIG. 15 the VSV-G envelope construct and FIG. 16 the gene transfer construct containing the ribozymes MAZI, SGIII and DGII.

Methods of delivery that may be used include encapsulation in drug delivery vehicles, especially; liposome, transduction by retroviral (including lentiviral) vectors and conjugation with cholesterol.

Drug delivery vehicles are effective for both systematic and topical administration. They can be designed to serve as a slow release reservoir, or to deliver their contents directly to the target cell. Some examples of such specialized drug delivery vehicles are liposomes, hydrogels, cyclodextrins, biodegradable nanocapsules, and bioadhesive microspheres.

Liposomes are preferred. They are hollow spherical vesicles composed of lipids arranged in a similar fashion as the lipids of the cell membrane. They have an internal aqueous space for entrapping water soluble compounds and range in size from 0.05 to several microns in diameter. Liposomes can deliver the DNA to cells, so that the nucleic acid remains biologically active.

They can easily be prepared by mixing the DNA with a liposome-forming lipid such as a dialkyl or diacylglycerol or phosphatidinylcholine, as known in the art of liposome formation. See J. J. Rossi et al. AIDS Research and Human Retroviruses 8, 183-189 (1992).

Liposome preparations useful in the invention comprise: (a) lipofectamine reagent (GIBCO BRL, Gaithersburg, Md. USA) containing a polycationic lipid molar ratio, (b) the cationic lipid, DDAB and DOPE, in a 2:1 ratio, R. Philip, Mol. Cell. Biol. 14, 2411-2418, (1994); and (c) DMRIE, optionally in combination with DOPE, e.g. in a 1:1 molar ratio (VICAL Corp. San Diego, Calif., USA). Newer liposomes, for example the serum-resistant cationic lipid GS 2888, J. G. Lewis et al., Proc. Natl. Acad. Sci. USA 93, 3176 (1996) and liposomes containing a polylysine/DNA complex, S. Li and L. Huang, J. Liposome Research 7, 63-75 (1997), can also be used.

Nanoparticles and hydrogel carriers have been developed for chemotherapeutic agents and protein-based pharmaceuticals, and consequently, can be adapted for ribozyme delivery for the purposes of the present invention.

Another delivery method is via T-cells. Compatible T-cells, preferably the patient's own are infected with ribozymal DNA of the invention, for example by electroporation and the patient is then infused with these cells. Electroporation of T-lymphocytes with DNA is described in Example 6 of PCT Publication WO 96/22638 (Gene Shears Pty Ltd.) and this method can be applied in the present invention.

The compositions for pharmaceutical use will normally contain a magnesium salt, preferably as buffered magnesium chloride, this being required for the function of the ribozyme. They may also contain a carrier or diluent, which can include a suspending or emulsifying agent.

Vector systems of this invention, preferably lentiviral vector systems, are preferably systemically administered, e.g. by an intravenous, subcutaneous, intraperitoneal, intranasal or intrathecal route. The dosage of ribozyme provided by the vector system will depend upon the disease indication and the route of administration but should be up to 200 mg/kg and usually at least 10 mg/kg of body weight/day. The posology will depend upon efficacy data from clinical trials.

The following Examples illustrate the invention. All DNA oligonucleotides used therein may be prepared by standard synthetic methods e.g. using solid phase synthesis by the phosphotriester method.

### MAZI Ribozymal DNA Cassette

A complete ribozymal DNA cassette was constructed having the nucleotide sequence SEQ ID NO: 4:

(143)
aaggtaccta atacgactca ctatagggcg
aaagcccggg acgactgatg agcgcgaaag
cgcgaaagag ccgtcgtgca cgcgaaagcg
tgcacctgat gaggccggaa aggccgaaac
ggctctttgg atcctctaga tt

It was made from a forward oligomer from positions 1 to 80 of the coding sequence and a reverse one from positions 143 to 58, using an oligonucleotide synthesizer. 26 bases at the 3′-ends of the oligomers were totally complementary to each other and the two strands were annealed. Their elongation to become a complete double strand was carried out with DNA polymerase on a PCR machine. The 143-long ds DNA cassette was cloned into pUC19 using KpnI and XbaI sites for the 5′-end and 3′-end respectively. Its sequence was confirmed by DNA sequencing. The cassette contains a T7 promoter, as the commercially available pUC19 does not contain this site.

5′ aaaggtacc taatacgactcactata gggcgaaagccc
KpnI T7 Promoter Stability Loop
gggacga ctgatga gcgcgaaagcgc gaa
MAZI stemI catalytic domain StemII Cat Dom
agagcc gtc gtgcacgcgaaagcgtgcac
StemIII overhang StemI for Sc ribozyme
ctgatga ggccggaaaggcc gaa
Sc. Cat. Dom Sc StemII Sc. Cat. Dom
acggctcttt ggatcc tctaga tt 3′
Sc StemIII XbaI

Starting from the 5′ end, first 9 bases constitute a KpnI restriction site, after which there are 17 bases which make the following T7 promoter sequence 12 bases fold up into a stem loop structure for stability, followed by a target specific region of 7 bases complementary to 1493-1486, making up the stem I. The catalytic domain ctgatga . . . gaa is sandwiching the intervening stem II sequence. The stem III structure is made up of next 5 bases being complementary to 1485-1480 along the HBV sequence in reverse. The last 3 bases provide the NUX site for the 3′ cis-acting ribozyme and is an overhang. The next 20 bases form the stem I of the cis-acting ribozyme, followed by catalytic domain bases with stem II in the middle. In the end there is stem III. In the end the last 14 bases make up two restriction sites for BamHI and XbaI enzymes. FIG. 1 shows a trans-acting MAZI ribozyme and FIG. 7 shows the structure of cis and trans acting MAZI ribozymes.

The ribozymal RNA was made by adding T7 polymerase in “Ribomax” solution, as described by Promega's “Protocols and Applications Guide” manual. “Ribomax” is a balanced salt solution containing the necessary magnesium ions for ribozymal activity. T7 polymerase triggers the T7 promoter to transcribe RNA from the DNA. Transcripts were isolated by using RNase-free DNase, followed by acid/phenol isolation of RNA, then ethanol precipitation. It was done as described in Molecular Cloning—A Laboratory Manual, cited above.

When the RNA was run on an agarose gel, the ribozyme before cleavage (103 bases FIG. 7) was clearly separated from the ribozyme after cleavage (50 bases, FIG. 1).

### MAZI Target Cleavage

HBV genomic DNA cloned into pcDNA3, was used (Molecular Cloning—A Laboratory Manual, cited above) to produce an RNA transcript of HBV.

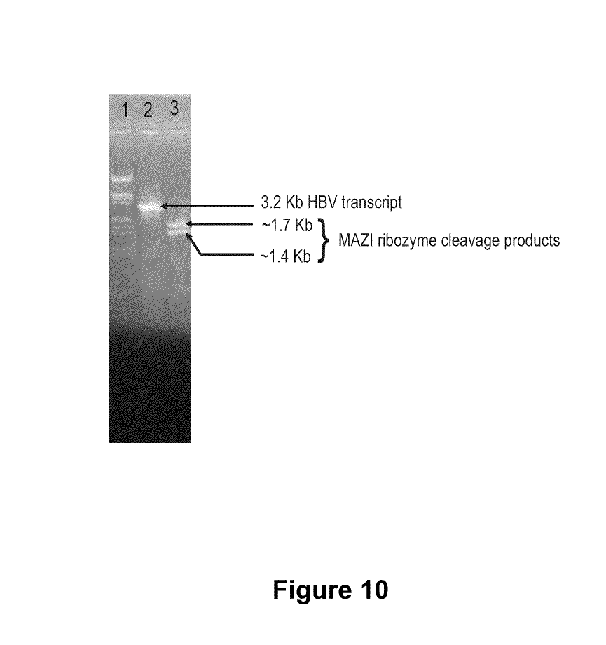

Ribozymes transcribed from the plasmid described in Section 2 above were incubated with the HBV RNA transcript at a molar ratio of 1 mole ribozyme to 1 moles of HBV RNA transcript. Within 4 hours of incubation at 37° C., total cleavage of the HBV RNA target was achieved. The RNA was run on 1% agarose gel containing ethidium bromide. Gel photographs taken under UV irradiation are presented in FIG. 10. The lane 1 consists of molecular weight markers, lane 2 consists of HBV transcript incubated with 24 mM MgCl2 for 4 hours and 37° C. and lane 3. HBV transcript incubated with equi-molar MAZI ribozyme transcript and 24 mM MgCl2 for 4 hours at 37° C. Thus it is clear that the MAZI ribozyme cleaves HBV RNA. Referring to FIG. 10, the right-hand lane shows the products after 4 hours of incubation, when the target RNA has been completely cleaved. The band representing uncleaved mRNA in FIG. 10 has disappeared and has been replaced by a band corresponding to the 3′-end of the cleaved product, at lower molecular weight. The fragment containing the 5′-end of the HBV RNA is visible at even lower molecular weight than in FIG. 10. There is no visible band containing MAZI ribozyme. This is because of the small size of the ribozyme molecule.

### SGIII Ribozymal DNA Cassette

A complete ribozymal DNA cassette was constructed having the nucleotide sequence SEQ ID NO: 5:

(137)
aattcgagct ctaatacgac tcactatagg
gcgaaagccc gatacactga tgagcgcgaa
agcgcgaaag ctgaggtcca cgtagaaata
cgtgctgatg aggacgaaag tccgaaacct
cagctttgaa ttcgataa

It was made from a forward oligomer from positions 1 to 80 of the coding sequence and a reverse one from positions 137 to 58, using an oligonucleotide synthesizer. 22 bases at the 3′-ends of the oligomers were totally complementary to each other and the two strands were annealed. Their elongation to become a complete double strand was carried out with DNA polymerase on a PCR machine. The 137-long ds DNA cassette was cloned into pUC19 using SacI and EcoRI sites for the 5′-end and 3′-end respectively. Its sequence was confirmed by DNA sequencing. The cassette contains a T7 promoter, as the commercially available pUC19 does not contain this site.

```
5′ aattcgagctc taatacgactcactata gggcgaaagccc
              SacI T7 Promoter Stability Loop
  gataca ctgatga gcgcgaaagcgc gaa
SGIII Stem I Cat. Dom Stem II Cat. Dom
agctgagg tc cacgtagaaatacgtg
SGIII StemIII overhang Sc StemI
ctgatga ggacgaaagtcc gaa
Sc Cat. Dom Sc. StemII Sc Cat. Dom
acctcagcttt gaattcgataa 3′
Sc. Stem III EcoRI
```

Starting from the 5′ end, first 9 bases constitute a SacI restriction site, after which there are 17 bases which make the T7 promoter sequence, 12 bases fold up into a stem loop structure for stability, followed by the target specific region of 5 bases complementary to 2017-2012, making up the stem I. The catalytic domain is sandwiching the stem II sequence. The stem III structure is made up of next 8 bases being complementary to 2010-2002 along the HBV sequence in reverse. The last two bases provide the NUX site for the 3′ cis-acting ribozyme and are an overhang. The next 16 bases form the stem I of the cis-acting ribozyme, followed by catalytic domain bases with stem II in the middle. In the end there is stem III. In the end the last 11 bases make up two restriction site for EcoRI enzyme.

The ribozymal RNA was made by adding T7 polymerase in “Ribomax” solution, as described by Promega's “Protocols and Applications Guide” manual. “Ribomax” is a balanced salt solution containing the necessary magnesium ions for ribozymal activity. T7 polymerase triggers the T7 promoter to transcribe RNA from the DNA. Transcripts were isolated by using RNase-free DNase, followed by acid/phenol isolation of RNA, then ethanol precipitation. It was done as described in Molecular Cloning—A Laboratory Manual, cited above. When the RNA was run on an agarose gel, the ribozyme before cleavage (102 bases FIG. 8) was clearly separated from the ribozyme after cleavage (51 bases, FIG. 2). RNA was shown by ethidium bromide contained in the gel (visualized under UV light).

### SGIII Target Cleavage

HBV genomic DNA cloned into pcDNA3, was used (Molecular Cloning—A Laboratory Manual, cited above) to produce an RNA transcript of HBV.

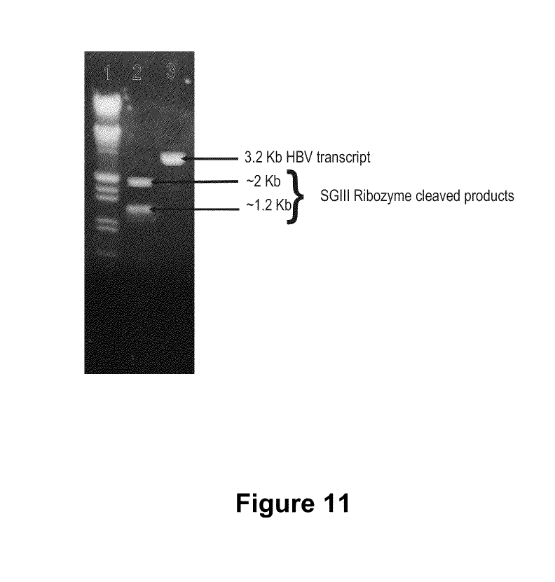

Ribozymes transcribed from the plasmid described in Section 2 above were incubated with the HBV RNA transcript at a molar ratio of 1 mole ribozyme to 1 moles of HBV RNA transcript. Within 4 hours of incubation at 37° C., total cleavage of the HBV RNA target was achieved. The RNA was run on 1% agarose gel containing ethidium bromide. Gel photographs taken under UV irradiation are presented in FIG. 11. The lane 1 consists of molecular weight markers, lane 2 consists of HBV transcript incubated with equi-molar SGIII ribozyme transcript and 24 mM MgCl₂ for 4 hours at 37° C. and lane 3 consists of HBV transcript incubated with 24 mM MgCl₂ for 4 hours and 37° C. Thus it is clear that the SGIII ribozyme cleaves HBV RNA. Referring to FIG. 11, the right-hand lane shows the products after 4 hours of incubation, when the target RNA has been completely cleaved. The band representing uncleaved mRNA in FIG. 11 has disappeared and has been replaced by a band corresponding to the 5′-end of the cleaved product, at lower molecular weight. The fragment containing the 3′-end of the HBV RNA is visible at even lower molecular weight than in FIG. 11. There is no visible band containing SGIII ribozyme. This is because of the small size of the ribozyme molecule.

### DGII Ribozymal DNA Cassette

A complete ribozymal DNA cassette was constructed having the nucleotide sequence SEQ ID NO: 6:

131
tatctagagg tacctaatac gactcactat
aaagcccggg ccctccaact gatgagcgcg
aaagcgcgaa atcctctcca atcctctcca
aggatcgaaa gatccgaaa gaggactgg
agctcgaatt c

It was made from a forward oligomer from positions 1 to 80 of the coding sequence and a reverse one from positions 131 to 51, using an oligonucleotide synthesizer. 19 bases at the 3′-ends of the oligomers were totally complementary to each other and the two strands were annealed. Their elongation to become a complete double strand was carried out with DNA polymerase on a PCR machine. The 131-long ds DNA cassette was cloned into pUC19 using KpnI and SacI sites for the 5′-end and 3′-end respectively. Its sequence was confirmed by DNA sequencing. The cassette contains a T7 promoter, as the commercially available pUC19 does not contain this site.

5′ TATACTAGAGGTACC TAATACGACTCACTATA GGGCGAAAGCCC
Kpn I T7 Promoter Stability Loop
UCCAA CUGAUGA GCGCGAAAGCGC GAA AGUCCUCU
Binding site Cat. Dom Stem II Cat Dom Stem III
C CACGUAGAAAUACGUG CUGAUGA GGAUCGAAAGAUCC
To overhang Sc Stem I Sc Cat Dom Sc Stem II
GAA AGAGGACUG GAGCTCGAATTC 3′
Sc Cat Dom Sc Stem III Sac I EcoRi

Starting from the 5′ end the first 15 bases contain a Kpn I restriction site, after which there are 17 bases which make the T7m promoter sequence, 12 bases fold up into a stem loop structure for stability, followed but target specific region of 5 bases complementary to 1672-1667, making up the stem I. The catalytic domain is sandwiching the stem II sequence. The stem III structure is made up of the next 8 bases being complementary to 1665-1658 along the HBV sequence in reverse. The last base provides the NUX site for the 3′ cis-acting ribozyme and is an overhang. The next 16 bases form the stem I of the cis-acting ribozyme, followed by catalytic domain bases with stem II in the middle. Stem III follows and at the end are restriction sites for Sac I and EcoRI.

The ribozymal RNA was made by adding T7 polymerase in “Ribomax” solution, as described by Promega's “Protocols and Applications Guide” manual. “Ribomax” is a balanced salt solution containing the necessary magnesium ions for ribozymal activity. T7 polymerase triggers the T7 promoter to transcribe RNA from the DNA.

Transcripts were isolated by using RNase-free DNase, followed by acid/phenol isolation of RNA, then ethanol precipitation. It was done as described in Molecular Cloning—A Laboratory Manual, cited above.

When the RNA was run on an agarose gel, the ribozyme before cleavage (103 bases) was clearly separated from the ribozyme after cleavage (48 bases, FIG. 3). RNA was shown by ethidium bromide contained in the gel (visualized under UV light).

### DGII Target Cleavage

HBV genomic DNA cloned into pcDNA3, was used (Molecular Cloning—A Laboratory Manual, cited above) to produce an RNA transcript of HBV.

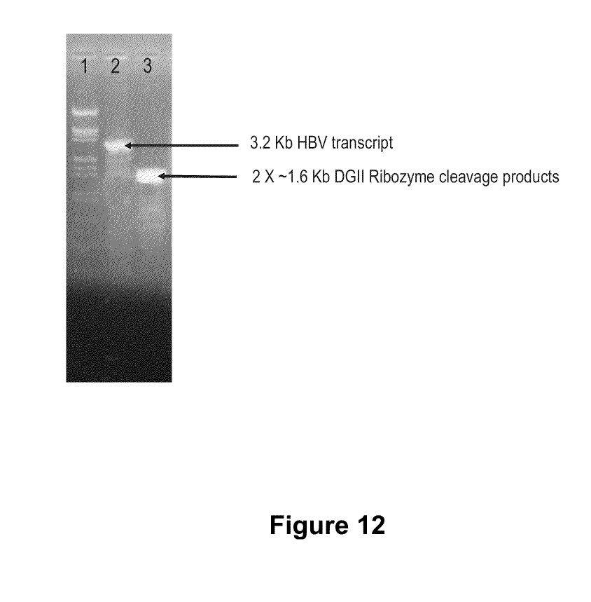

Ribozymes transcribed from the plasmid described in Section 2 above were incubated with the HBV RNA transcript at a molar ratio of 1 mole ribozyme to 1 moles of HBV RNA transcript. Within 4 hours of incubation at 37° C., total cleavage of the HBV RNA target was achieved. The RNA was run on 1% agarose gel containing ethidium bromide. Gel photographs taken under UV irradiation are presented in FIG. 12. Lane 1 consists of molecular weight markers. Lane 2 consists of a 3.2 kb HBV transcript. Lane 3 consists of the HBV transcript incubated with equi-molar DGII ribozyme for 4 hours. Thus it is clear that the DGII ribozyme cleaves HBV RNA as lane 3 shows that the 3.2 kb HBV transcript has been cleaved into 2 ˜1.6 kb cleavage products due to incubation with DGII. There is no visible band containing DGII ribozyme. This is because of the small size of the ribozyme molecule.

### Lentiviral Delivery of MAZI, SGIII and DGII

A lentiviral delivery system for ribozymes MAZI, SGIII and DGII was assembled with the packaging and envelope constructs shown in FIGS. 14, 15 and 16, according to standard methods.

To provide a model for the demonstration of HBV genome cleavage by lentivirally-delivered MAZI, SGIII and DGII ribozymes, an in vitro system was constructed in which RNA from DNA plasmids expressing portions of the HBV genome was expressed in cultured cells. These transfected cells were then transduced with a lentivirus expressing the MAZI, SGIII and DGII ribozymes, in order to cleave the HBV genome.

FIG. 16 shows plasmids pHBV1 and pHBV2, which express sequences 1-1579 and 1500-3182 of the HBV genome respectively. H293T cells (5.0 E5) were transfected (Superfect, QIAGEN UK) with pHBV1 and HBV2 vectors and after 72 hours, the transfection was repeated. Twenty four hours post re-transfection, the cells (1.0×10⁶) were transduced with Lenti-HBVRZ virus (MOI=100). Forty eight hours post transduction, the cells were harvested for RNA and DNA.

PCR primers were designed such that resulting PCR products included the cleavage sites of MAZI, SGIII and DGII. These primers were designed based on HBV sequence HBVAYGEN, Accession No:Y07587 and are shown below together with the lengths of the PCR products produced by these primer pairs on HBV template.

| MAZI Primers | |
|---|---|
| 1 | GGACGTCCTTTGTTTACGTCCCGT |
| | 1,414: GCGCG GGACGTCCTTTGTTTACGTCCCGT CGGCG |
| 2/Rev | GACCACGGGGCGCACCTCTCTTTA |
| | 1,517: CGACC GACCACGGGGCGCACCTCTCTTTA CGCGG |
| Length of PCR Product = 127 | |
| DGII Primers | |
| 1 | CACGTCGCATGGAGACCACCGT |
| | 1,602: CTCTG CACGTCGCATGGAGACCACCGT GAACG |
| 2/Rev | CTGCAATGTCAACGACCGACCTTG |
| | 1,677: ACTCT CTGCAATGTCAACGACCGACCTTG AGGCA |
| Length of PCR Product = 99 | |
| SGIII Primers | |
| 1 | ACGTGATCTTCTAGATACCGCCTC |
| | 1,983: TCAGT ACGTGATCTTCTAGATACCGCCTC AGCTC |
| 2/Rev | AGCTACCTGGGTGGGTGGTAATTT |
| | 2,106: ACTCT AGCTACCTGGGTGGGTGGTAATTT GGAAG |
| Length of PCR Product = 147 | |
| In summary, primer pairs were as follows: | |
| MAZI primers | |
| 1 | GGACGTCCTTTGTTTACGTCCCGT |
| 2 | TAAAGAGAGGTGCGCCCCGTGGTC |
| DGII primers | |
| 1 | CACGTCGCATGGAGACCACCGT |
| 2 | CAAGGTCGGTCGTTGACATTGCAG |
| SGIII primers | |
| 1 | ACGTGATCTTCTAGATACCGCCTC |
| 2 | AAATTACCACCCACCCAGGTAGCT |

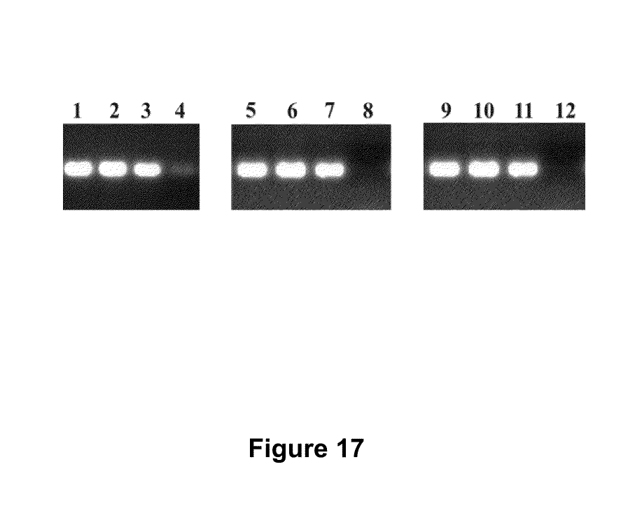

The DNA and RNA from H293T cells expressing the HBV target sequences (from pHBV1 and pHBV2) with and without transduction by Lenti-HBVRZ was analyzed by PCR and RTPCR respectively, using the above primers. FIG. 17 is a photograph from an agarose gel showing these PCR products. Lanes 1 to 4 show that in control cells transfected with pHBV1, the MAZI target sequence is present in the DNA and RNA (Lanes 1 and 2). Where transduced with Lenti-HBVRZ, the DNA is still present, but the RNA translated from this plasmid is very much reduced as it has been cleaved by MAZI (Lanes 3 and 4). Lanes 5 to 8 show that in control cells transfected with pHBV2, the DGII target sequence is present in the DNA and RNA (Lanes 5 and 6). Where transduced with Lenti-HBVRZ, the DNA is still present, but the RNA translated from this plasmid is now absent as it has been cleaved by DGII (Lanes 7 and 8). Lanes 9 to 12 show that in control cells transfected with pHBV2, the DGII target sequence is present in the DNA and RNA (Lanes 9 and 10). Where transduced with Lenti-HBVRZ, the DNA is still present, but the RNA translated from this plasmid is now absent as it has been cleaved by DGII (Lanes 11 and 12).

These results clearly demonstrate that the ribozymes MAZI, SGIII and DGII are active in cleaving target HBV RNA in cells when delivered by a lentiviral vector.

TABLE 1
HBV sequence (Accession No Y07587.1 GI: 1514493)

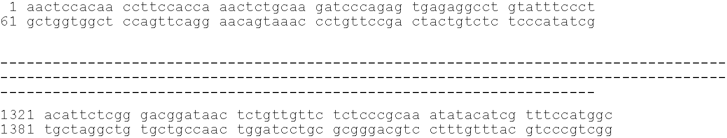

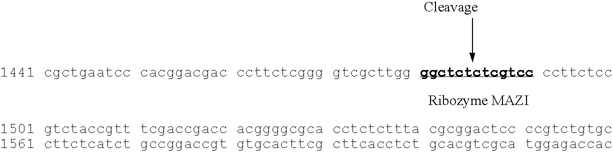

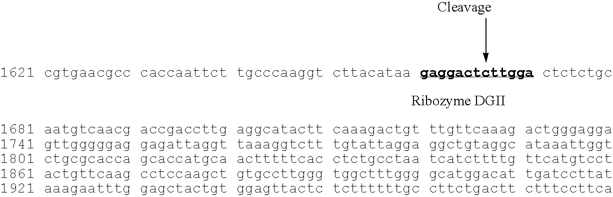

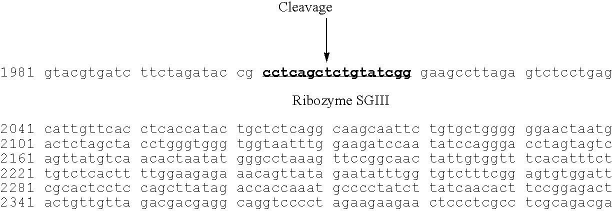

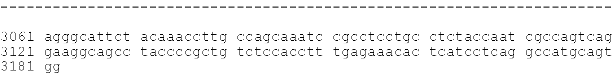

Sequences

SEQ ID NO: 1

ggcucucucguccc

SEQ. ID NO: 2

ccucagcucuguaucg

SEQ ID NO: 3

gaggacucuugga

SEQ ID NO: 4

aaggtaccta atacgactca ctatagggcg aaagcccggg
acgactgatg agcgcgaaag cgcgaaagag ccgtcgtgca
cgcgaaagcg tgcacctgat gaggccggaa aggccgaaac
ggctctttgg atcctctaga tt

SEQ ID NO: 5

aattcgagct ctaatacgac tcactatagg gcgaaagccc
gatacactga tgagcgcgaa agcgcgaaag ctgaggtcca
cgtagaaata cgtgctgatg aggacgaaag tccgaaacct
cagctttgaa ttcgataa

SEQ ID NO: 6

tatctagagg tacctaatac gactcactat aaagcccggg
ccctccaact gatgagcgcg aaagcgcgaa atcctctcca
atcctctcca aggatcgaaa gatccgaaa gaggactgg
agctcgaatt c

1. Ribozymal DNA transcribable to an RNA having a catalytic sequence in two parts, including an intervening, stabilizing stem having the sequence GCGCGAAAGCGC (SEQ ID NO:38) located between said two parts, wherein said RNA will cleave HBV RNA at a CUC site.

2. (canceled)

3. The ribozymal DNA according to claim 1, having a nucleotide sequence selected from the group consisting of SEQ ID NO: 4 (MAZ1), SEQ ID NO:5 (SGIII) and SEQ ID NO:6 (DGII).

4-5. (canceled)

6. The ribozymal DNA according to claim 1, which is transcribable to RNA which binds to an HBV recognition site having a nucleotide sequence selected from the group consisting of GGCUCUCUCGUCCC (MAZ1) (SEQ ID NO:1), CCUCAGCUCUGUAUCG (SGIII) (SEQ ID NO:2) and GAGGACUCUUGGA (DGII) (SEQ ID NO:3).

7-9. (canceled)

10. The ribozymal DNA according to claim 1, which, when transcribed to RNA, will cleave at three sites in a target RNA sequence present in HBV RNA and which contains recognition sequences as follows (5′ to 3′):
(SEQ ID NO:42)
GGCTCTCTCGTCCC for ribozyme MAZI
(SEQ ID NO:43)
CCTCAGCTCTGTATCG for ribozyme SGIII
(SEQ ID NO:41)
GAGGACTCTTGGA for ribozyme DGII.

11. A vector system comprising at least one DNA vector, the at least one DNA vector containing a target-cleaving ribozymal DNA sequence under control of a promoter effective in human cells and which, upon transcription to RNA will cleave the RNA transcribed from a HBV target genome at CUC sites therein.

12. The vector system according to claim 11, containing at least one target-cleaving ribozymal sequence for one or more of the ribozymes MAZI, DGII and SGIII cleaving mRNA transcribed from HBV target genome.

13. The vector system according to claim 11, comprising at least two DNA vectors, wherein a first vector contains a first promoter that allows gene expression in human cells, operably linked to a gene which is expressible to produce an activator protein capable of acting on a second promoter, wherein a second vector contains the second promoter operably linked to one or more of target-cleaving ribozymal DNA sequences for one or more RNA sequences selected from the group consisting of MAZI RNA, DGII RNA, and SGIII RNA to target HBV RNA.

14. The vector system according to claim 13, comprising at least three DNA vectors, wherein the second vector contains target-cleaving ribozymal DNA for MAZI, and wherein a third vector contains target-cleaving ribozymal DNA for SGIII cleaving mRNA transcribed from the targeted HBV RNA.

15. The vector system according to claim 14, comprising at least four DNA vectors, wherein the second vector contains target-cleaving ribozymal DNA for MAZI, the third vector contains target-cleaving ribozymal DNA for SGIII, and wherein a fourth vector contains target-cleaving ribozymal DNA for DGII cleaving mRNA transcribed from the targeted HBV RNA.

16. (canceled)

17. The vector system according to claim 13, wherein the second promoter is a T7 polymerase promoter and the activator protein is a T7 polymerase.

18-19. (canceled)

20. The vector system according to claim 11, wherein the ribozymal DNA sequence further comprises, downstream of the target-cleaving ribozymal sequence, a 3′-autocatalytic hammerhead ribozymal DNA sequence, so that when the ribozymal DNA is transcribed to RNA, it has a representable form as a double hammerhead, having first and second stems of a target-cleaving ribozyme which targets HBV RNA and first and second stems of 3′-autocatalytic ribozyme.

21-22. (canceled)

23. The vector system acid according to claim 20, wherein the target-cleaving ribozyme sequence comprises in order (5′ to 3′):
a first structure-stabilizing stem loop;
a first target-recognition sequence;
a first catalytic sequence;
a second structure-stabilizing stem loop;
a second catalytic sequence; and
a second target-recognition sequence.

24. The vector system according to claim 11, wherein the target-cleaving ribozymal DNA sequence, when transcribed to RNA, will cleave a target RNA sequence present in HBV RNA, and which contains recognition sequences (5′ to 3′):
(SEQ ID NO:42)
GGCTCTCTCGTCCC for ribozyme MAZI
(SEQ ID NO:43)
CCTCAGCTCTGTATCG for ribozyme SGIII
(SEQ ID NO:41)
GAGGACTCTTGGA for ribozyme DGII.

25. A lentivirus containing a vector system according to claim 11.

26. A pharmaceutically acceptable carrier containing ribozymal DNA according to claim 1, a vector system defined in claim 11, or a lentivirus according to claim 25.

27. The carrier according to claim 26 in the form of liposomes.

28. (canceled)

29. A method of treating a disease associated with HBV infection, comprising administering the ribozymal DNA according to claim 1, a vector system according to claim 11, or a lentivirus according to claim 25 to an individual in need thereof.

30-31. (canceled)

32. Ribozymal DNA comprising: (1) a target-cleaving hammerhead ribozymal DNA sequence under control of a promoter effective in human cells and which, upon transcription to RNA, will cleave mRNA transcribed from a target gene encoding an HBV genome, and downstream thereof; and (2) a 3′-autocatalytic hammerhead ribozymal DNA sequence, so that when the ribozymal DNA is transcribed to RNA, it has a form represented as a double hammerhead, having first and second stems of a target-cleaving ribozyme which targets HBV RNA and first and second stems of 3′-autocatalytic ribozyme, together with a common third system joining the two hammerheads.

33. (canceled)


SEQUENCE LISTING (43 sequences)

SEQ ID NO: 1 - RNA, 14 nt, Hepatitis B virus
ggcucucucguccc

SEQ ID NO: 2 - RNA, 16 nt, Hepatitis B virus
ccucagcucuguaucg

SEQ ID NO: 3 - RNA, 13 nt, Hepatitis B virus
gaggacucuugga

SEQ ID NO: 4 - DNA, 142 bp, Artificial Sequence
  Synthetic sequence MAZI ribozymal DNA cassette
aaggtacctaatacgactcactatagggcgaaagcccgggacgactgatgagcgcgaaagcgcgaaagagccgtcgtgcacgcgaaagcgtgcacctgatgaggccggaaaggccgaaacggctctttggatcctctagatt

SEQ ID NO: 5 - DNA, 138 bp, Artificial Sequence
  Synthetic sequence SGIII ribozymal DNA cassette
aattcgagctctaatacgactcactatagggcgaaagcccgatacactgatgagcgcgaaagcgcgaaagctgaggtccacgtagaaatacgtgctgatgaggacgaaagtccgaaacctcagctttgaattcgataa

SEQ ID NO: 6 - DNA, 129 bp, Artificial Sequence
  Synthetic sequence DGII ribozymal DNA cassette
tatctagaggtacctaatacgactcactataaagcccgggccctccaactgatgagcgcgaaagcgcgaaatcctctccaatcctctccaaggatcgaaagatccgaaagaggactggagctcgaattc

SEQ ID NO: 7 - RNA, 50 nt, Artificial Sequence
  Synthetic sequence Target-cleaving ribozyme sequence for MAZI ribozyme
gggcgaaagcccgggacgacugaugagcgcgaaagcgcgaaagagccguc

SEQ ID NO: 8 - RNA, 51 nt, Artificial Sequence
  Synthetic sequence Target-cleaving ribozyme sequence for SGIII ribozyme
gggcgaaagccccgauacacugaugagcgcgaaagcgcgaaagcugagguc

SEQ ID NO: 9 - RNA, 48 nt, Artificial Sequence
  Synthetic sequence Target-cleaving ribozyme sequence for DGII ribozyme
gggcgaaagcccuggaacugaugagcgcgaaagcgcgaaaguccucuc

SEQ ID NO: 10 - RNA, 48 nt, Artificial Sequence
  Synthetic sequence DGII ribozyme
gggcgaaagcccuccaacugaugagcgcgaaagcgcgaaaguccucuc

SEQ ID NO: 11 - RNA, 104 nt, Artificial Sequence
  Synthetic sequence MAZI ribozyme combined with an autocatalytic ribozyme in a trans/cis double ribozyme
gggcgaaagcccgggacgacugaugagcgcgaaagcgcgaaagagccgucgugcacgcgaaagcgugcaccugaugaggccggaaacggccgaaacggcucuuu

SEQ ID NO: 12 - RNA, 100 nt, Artificial Sequence
  Synthetic sequence SGIII ribozyme combined with an autocatalytic ribozyme in a trans/cis double ribozyme
gggcgaaagccccgauacacugaugagcgcgaaagcgcgaaagcugagguccacguagaaauacgugcugaugaggacgaaaguccgaaaccucagcuuu

SEQ ID NO: 13 - RNA, 97 nt, Artificial Sequence
  Synthetic sequence DGII ribozyme combined with an autocatalytic ribozyme in a trans/cis double ribozyme
gggcgaaagcccuccaacugaugagcgcgaaagcgcgaaaguccucuccacguagaaauacgugcugaugaggaucgaaagauccgaaagaggacug

SEQ ID NO: 14 - DNA, 28 bp, Artificial Sequence
  Forward PCR primer
acgaattccatggacacgattaacatcg

SEQ ID NO: 15 - DNA, 28 bp, Artificial Sequence
  Reverse PCR primer
atataaggatccttacgcgaacgcgaac

SEQ ID NO: 16 - DNA, 28 bp, Artificial Sequence
  Forward PCR primer
gctctagaccacaacggtttccctctag

SEQ ID NO: 17 - DNA, 28 bp, Artificial Sequence
  Reverse PCR primer
cagcttcctttcgggctttgttagcagc

SEQ ID NO: 18 - DNA, 143 bp, Artificial Sequence
  Synthetic sequence MAZI Ribozymal DNA cassette
aaaggtacctaatacgactcactatagggcgaaagcccgggacgactgatgagcgcgaaagcgcgaaagagccgtcgtgcacgcgaaagcgtgcacctgatgaggccggaaaggccgaaacggctctttggatcctctagatt

SEQ ID NO: 19 - DNA, 141 bp, Artificial Sequence
  Synthetic sequence DGII Ribozymal DNA cassette
tatactagaggtacctaatacgactcactatagggcgaaagcccuccaacugaugagcgcgaaagcgcgaaaguccucuccacguagaaauacgugcugaugaggaucgaaagauccgaaagaggacuggagctcgaattc

SEQ ID NO: 20 - DNA, 24 bp, Artificial Sequence
  Synthetic sequence MAZI primer 1
ggacgtcctttgtttacgtcccgt

SEQ ID NO: 21 - DNA, 34 bp, Hepatitis B virus
gcgcgggacgtcctttgtttacgtcccgtcggcg

SEQ ID NO: 22 - DNA, 24 bp, Artificial Sequence
  Synthetic sequence MAZI primer 2 / Rev
gaccacggggcgcacctctcttta

SEQ ID NO: 23 - DNA, 34 bp, Hepatitis B virus
cgaccgaccacggggcgcacctctctttacgcgg

SEQ ID NO: 24 - DNA, 22 bp, Artificial Sequence
  Synthetic sequence DGII primer 1
cacgtcgcatggagaccaccgt

SEQ ID NO: 25 - DNA, 32 bp, Hepatitis B virus
ctctgcacgtcgcatggagaccaccgtgaacg

SEQ ID NO: 26 - DNA, 24 bp, Artificial Sequence
  Synthetic sequence DGII primer 2 / Rev
ctgcaatgtcaacgaccgaccttg

SEQ ID NO: 27 - DNA, 34 bp, Hepatitis B virus
actctctgcaatgtcaacgaccgaccttgaggca

SEQ ID NO: 28 - DNA, 24 bp, Artificial Sequence
  Synthetic sequence SGIII primer 1
acgtgatcttctagataccgcctc

SEQ ID NO: 29 - DNA, 34 bp, Hepatitis B virus
tcagtacgtgatcttctagataccgcctcagctc

SEQ ID NO: 30 - DNA, 24 bp, Artificial Sequence
  Synthetic sequence SGIII primer 2 / Rev
agctacctgggtgggtggtaattt

SEQ ID NO: 31 - DNA, 34 bp, Hepatitis B virus
actctagctacctgggtgggtggtaatttggaag

SEQ ID NO: 32 - DNA, 24 bp, Artificial Sequence
  Synthetic sequence MAZI primer 2
taaagagaggtgcgccccgtggtc

SEQ ID NO: 33 - DNA, 24 bp, Artificial Sequence
  Synthetic sequence DGII primer 2
caaggtcggtcgttgacattgcag

SEQ ID NO: 34 - DNA, 24 bp, Artificial Sequence
  Synthetic sequence SGIII primer 2
aaattaccacccacccaggtagct

SEQ ID NO: 35 - DNA, 120 bp, Hepatitis B virus
aactccacaaccttccaccaaactctgcaagatcccagagtgagaggcctgtatttccctgctggtggctccagttcaggaacagtaaaccctgttccgactactgtctctcccatatcg

SEQ ID NO: 36 - DNA, 1080 bp, Hepatitis B virus
acattctcgggacggataactctgttgttctctcccgcaaatatacatcgtttccatggctgctaggctgtgctgccaactggatcctgcgcgggacgtcctttgtttacgtcccgtcggcgctgaatcccacggacgacccttctcggggtcgcttggggctctctcgtccccttctccgtctaccgtttcgaccgaccacggggcgcacctctctttacgcggactccccgtctgtgccttctcatctgccggaccgtgtgcacttcgcttcacctctgcacgtcgcatggagaccaccgtgaacgcccaccaattcttgcccaaggtcttacataagaggactcttggactctctgcaatgtcaacgaccgaccttgaggcatacttcaaagactgtttgttcaaagactgggaggagttgggggaggagattaggttaaaggtctttgtattaggaggctgtaggcataaattggtctgcgcaccagcaccatgcaactttttcacctctgcctaatcatcttttgttcatgtcctactgttcaagcctccaagctgtgccttgggtggctttggggcatggacattgatccttataaagaatttggagctactgtggagttactctcttttttgccttctgacttctttccttcagtacgtgatcttctagataccgcctcagctctgtatcgggaagccttagagtctcctgagcattgttcacctcaccatactgctctcaggcaagcaattctgtgctggggggaactaatgactctagctacctgggtgggtggtaatttggaagatccaatatccagggacctagtagtcagttatgtcaacactaatatgggcctaaagttccggcaactattgtggtttcacatttcttgtctcacttttggaagagaaacagttatagaatatttggtgtctttcggagtgtggattcgcactcctccagcttatagaccaccaaatgcccctatcttatcaacacttccggagactactgttgttagacgacgaggcaggtcccctagaagaagaactccctcgcctcgcagacga

SEQ ID NO: 37 - DNA, 122 bp, Hepatitis B virus
agggcattctacaaaccttgccagcaaatccgcctcctgcctctaccaatcgccagtcaggaaggcagcctaccccgctgtctccacctttgagaaacactcatcctcaggccatgcagtgg

SEQ ID NO: 38 - DNA, 12 bp, Hepatitis B virus
gcgcgaaagcgc

SEQ ID NO: 39 - DNA, 13 bp, Hepatitis B virus
ggctctctcgtcc

SEQ ID NO: 40 - DNA, 17 bp, Hepatitis B virus
cctcagctctgtatcgg

SEQ ID NO: 41 - DNA, 13 bp, Hepatitis B virus
gaggactcttgga

SEQ ID NO: 42 - DNA, 14 bp, Hepatitis B virus
ggctctctcgtccc

SEQ ID NO: 43 - DNA, 16 bp, Hepatitis B virus
cctcagctctgtatcg